(19) United States
(12) Patent Application Publication
(10) Pub. No.: US 2026/0109935 A1
(43) Pub. Date: Apr. 23, 2026

(54) METHOD FOR PERFORMING A BIOPROCESS ON LIQUID IMMUNE CELL CULTURES COMPRISING A SEQUENCE OF PROCESSING STEPS

(71) Applicant: The Automation Partnership (Cambridge) Ltd, Royston, Hertfordshire (GB)

(72) Inventors: Adrian Stacey, Cambridge (GB); Fanny Prouté, Cambridge (GB)

(21) Appl. No.: 19/114,267

(22) PCT Filed: Sep. 21, 2022
(86) PCT No.: PCT/IB2022/000491

§ 371 Date: Mar. 21, 2025

(51) Int. Cl. C12M 1/00; C12M 1/34; C12M 3/00

(52) U.S. Cl. CPC C12M 47/04; C12M 23/40; C12M 23/42; C12M 41/12

(57) ABSTRACT

Method for performing a bioprocess on liquid immune cell cultures comprising a sequence of processing steps, wherein the bioprocess is being performed on an integrated bioprocessing system, wherein the sequence of processing steps comprises at least one unit operation, which is being performed within a unit operation station of the integrated bioprocessing system. Cartridges, that have been preconfigured with a cartridge fluidic structure with respect to the respective unit operation, are being stored in a cartridge storage unit of the unit operation station and that for performing the respective unit operation, one of the cartridges is being transported from a storage transfer location in a cartridge storage unit of the unit operation station to a drive location of a cartridge drive unit of the unit operation station by a local transport mechanism of the unit operation station and is being brought into operative coupling with the cartridge drive unit.

### CROSS-REFERENCE TO RELATED APPLICATIONS

This application is a national stage application under 35 U.S.C. 371 of International Patent Application Serial No. PCT/IB2022/000491, entitled “Method for Performing a Bioprocess on Liquid Immune Cell Cultures Comprising a Sequence of Processing Steps,” filed Sep. 21, 2022, the disclosure of which is incorporated herein by reference.

### FIELD OF THE TECHNOLOGY

Various embodiments relate to a method for performing a bioprocess on liquid immune cell cultures comprising a sequence of processing steps, to a unit operation station of an integrated bioprocessing system for performing at least one bioprocess on liquid immune cell cultures and to an integrated bioprocessing system for performing a bioprocess on liquid immune cell cultures.

### SUMMARY

The term “bioprocess” presently represents a biotechnological process, in particular a biotechnological process involving the use of immune cell cultures. One or more processing steps might be performed on each immune cell culture. Hence, a bioprocess in this sense might refer to a manufacturing process that involves a sequence of processing steps performed on an immune cell culture which ultimately will lead to a final product. Additionally or alternatively, the term “bioprocess” refers to a single processing step or a number of processing steps that are applied to an immune cell culture to optimize or improve a certain processing step and/or a sequence of processing steps.

The method in question for performing a bioprocess on immune cell cultures may be applied in various fields of biotechnology and for different kinds of bioprocesses. Exemplarily, the proposed method may be used in the area of cell and gene therapy, including allogenic or autologous production of genetically modified immune cells. Exemplarily, the method may be applied to manufacture autologous T cells that are modified to express a chimeric antigen receptor (CAR). These cells might be used for the treatment of various types of hematologic malignancies, including different types of leukemia (blood cancer).

For biotechnological processes involving the use of liquid immune cell cultures, process flexibility is especially relevant. The starting material might be quite heterogeneous regarding its composition, for example because each patient is in a different condition (e.g., in terms of disease progression or in terms of genetic make-up and history of their immune system). The starting material might also be heterogenous regarding its composition because of immune cell cultures of different donors being combined into one initial starting material. Hence, depending on the source of the liquid immune cell culture, various parameters, including for example the type and concentration of different cell types, the overall cell viability and vitality and/or the amount and type of impurities within the liquid immune cell culture might vary. Additionally, depending on the bioprocess to be performed, the cell culture might comprise different types of immune cells in different amounts (e.g., T cells, dendritic cells or immune cells in different developmental stages including naïve T cells). Partly because of the reasons mentioned above, the sequence of processing steps performed on each liquid immune cell culture will need to be adjusted to the individual features of the cell culture. In addition, certain steps of the bioprocess might need to be flexibly adapted and tailored to each individual cell culture. For example, the type of genetic modification of the immune cell culture might be different as patients may respond differently to a certain genetic modification. Accordingly, the bioprocess will differ in the way genetic modification is performed. Also exemplarily, a bioprocess involving the genetic modification of an immune cell culture will require a sequence of processing steps that is different from the sequence of processing steps required in a bioprocess not involving the genetic modification of an immune cell culture. This requires that a method for performing a bioprocess on liquid immune cell cultures can be adapted in a flexible manner.

While process flexibility is very important, cost-efficiency is also an important aspect during processing of liquid immune cell cultures. A reason for this is that the requirements from regulatory authorities are very demanding. For example, operators involved in the process will need to be extensively trained, especially when performing manual processes. In addition, sterility must be kept during the manufacturing process, because the processed immune cells need to be viable and must not include contaminants when being administered to the recipient. Partly for the reasons mentioned, the overall costs for each bioprocess performed are very high. Additionally, in approaches, where one single device is used to sequentially perform all processing steps on an initial immune cell culture, the facility footprint is usually large, as one device is required to process one immune cell culture at a time. Further, the throughput is lowered, as only one liquid immune cell culture is processed within one device. Also, as the sequence of processing steps is typically predefined, for example by the tubing set installed, adaptation of such devices to changes in the sequence or type of processing steps performed is rather demanding. Nevertheless, these end-to-end systems provide a system wherein all media are kept within a closed inner volume, which supports maintenance of sterility.

Cost-efficiency is particularly related to the operating costs; hence, it is desirable to enable the processing of more than one immune cell culture in parallel. This also increases the throughput, which is important given the fact that processing of a single initial immune cell culture might take several days or even weeks. However, when processing more than one immune cell culture in parallel, it needs to be ensured that no cross-contamination of different immune cell cultures occurs.

Another important aspect is redundancy, which also relates to process robustness and process reliability. As mentioned above and especially when applied for cancer treatment, processing of an immune cell culture might take considerable time with patients typically requiring treatment urgently. Hence, it needs to be ensured that the process does not fail. Process failure might result in the process having to be repeated, which would require further time and/or additional starting material needed. However, especially in autologous approaches, it might not be possible to obtain any further starting material from the patient.

WO 2021/212124 A1, that builds the basis of various embodiments is related to a method. This reference discloses a method for the modular and parallelized processing of liquid immune cell cultures on an integrated bioprocessing system. The method comprises a sequence of processing steps, which are being performed within specific unit operation stations of the bioprocessing system. Although the bioprocessing system shows a certain degree of standardization in the form of a rack receiving all components necessary to perform the unit operations, the unit operation stations as such are each highly customized with regard to the unit operation to be performed.

Accordingly, the configuration of each complete unit operation station is dependent on the type of unit operation performed at the respective unit operation station. Consequently, also the infrastructure (e.g. reservoirs for raw materials and/or waste materials) provided at a unit operation station depends on the unit operation that is being performed at the respective unit operation station. Additionally, the interaction between a transport system and each unit operation station depends on the type of unit operation performed at the respective unit operation station. With the resulting, high degree of customization regarding the unit operation stations, the flexibility regarding the definition of the process steps is high; however, the potential for increasing efficiency by automation stays comparably low.

Various embodiments are based on the problem of improving the known method such that the potential for automation is increased without compromising the flexibility with respect to the definition of process steps.

The above-noted object is solved by various features provided herein.

A realization of various embodiments is that each unit operation comprises unit operation steps, that are highly individual, and unit operation steps, that are always the same. It has been found, that the latter unit operation steps are mainly regarding the transport and connection of fluidic structures and mechanically and/or fluidically driving the respective unit operation. Therefore, one important aspect of various embodiments is to provide individualized cartridges, which are all being stored in one and the same cartridge storage unit. For performing any of the unit operations, a standard routine is to be followed, which comprises the transport of the individualized cartridge from the cartridge storage unit to a cartridge drive unit and the operative coupling of the cartridge with the cartridge drive unit. For this transport, a local transport mechanism, which is assigned to the unit operation station, is provided.

In detail, it is proposed that cartridges, that have been preconfigured with a cartridge fluidic structure with respect to at least one unit operation, are being stored in a cartridge storage unit of the unit operation station and that for performing a unit operation, in the standard routine, one of the cartridges is being transported from a storage transfer location in the cartridge storage unit of the unit operation station to a drive location of a cartridge drive unit of the unit operation station by a local transport mechanism of the unit operation station and is being brought into operative coupling with the cartridge drive unit.

All in all the unit operation stations each provide a number of standard functions, such as the storage function, the transport function and the driving function for the cartridges, which allows for a high degree of automation. At the same time, the cartridges may be configured highly individually, such that the flexibility regarding the performance of tailored unit operations and processing steps on a particular immune cell culture is not compromised.

The term “operative coupling” is to be understood in a broad way. In various embodiments, it means the functional engagement of the cartridge drive unit to the cartridge. Hence, the term “coupling” may refer to the physical engagement between both components. Additionally or alternatively, it may also refer to an indirect connection (e.g., by magnetic actuation) established between two components. Essential is that the cartridge drive unit actuates the respective element of the cartridge.

The proposed solution allows for the standardization of a wide range of components as such. According to various embodiments, the cartridges may be standardized with the exception of the respective cartridge fluidic structures. This may regard a cartridge carrier for example, which allows for standardized transport. This again makes it possible to simplify the respective local transport mechanism.

Various embodiments are directed to a variant for transferring liquids to and from a cartridge. For this, standardized receptacles can be used, that may be transported to transfer locations of the respective unit operation station by a global transport mechanism. The advantage here is that the transfer locations may be standardized in terms of transfer location and/or geometry and, again, that the global transport mechanism may be of simplified construction.

The integrated bioprocessing system may comprise a number of unit operation stations. This allows several unit operations to be carried out in parallel, which increases the throughput of the integrated bioprocessing system.

Not only the cartridges as noted above, but also the unit operation stations may be standardized according to various embodiments. This leads to the possibility of scaling the integrated bioprocessing system and with it the proposed method up and down as desired without the need of changing the constructional concept.

All liquids involved in a unit operation are being kept within a closed inner volume. This ensures that the sterility of all liquids is maintained. It further ensures that no cross-contamination between different immune cell cultures occurs. This is especially important when multiple immune cell cultures are processed in parallel. Further, the requirements of the environment regarding sterility are largely reduced. The term “closed inner volume” presently means, that all noted liquids are being maintained and guided within a volume, that is separated from atmosphere. The term “atmosphere” presently represents the volume outside the above noted, closed inner volume. This ensures to maintain sterility within the closed inner volume. As explained above, maintaining sterility is a critical aspect in the manufacturing process. The closed inner volume may easily be realized, if at least part of the liquid handling involved in the respective unit operation is being performed within tubes.

According to various embodiments, the tubes for liquid transfer are being connected in a closed connection process, such that the liquids stay separated from atmosphere as far as possible. For this, the application of a welding system, which is further detailed in various embodiments, which can allow for automation with little constructional effort and which makes it unnecessary to provide complicated and potentially expensive sterile connectors or the like.

Providing the unit operation stations with an assigned waste unit makes it possible to store multiple used cartridges in the form of a cartridge waste storage, which is part of the respective unit operation station. This gives flexibility in view of the parallel performance of various bioprocesses, as the used cartridge does not have to be transported by the global transport mechanism right away.

The simultaneous performance of at least two bioprocesses is subject of various embodiments, aiming for an increase of throughput of the integrated bioprocessing system. In various embodiments, an electronic process control is provided, which coordinates those bioprocesses. The electronic process control may comprise a hardware, on which a control software is running. The control software can include optimization strategies to run two or more bioprocesses simultaneously in view of predefined optimization strategies regarding time efficiency, quality, cost efficiency or the like.

Various embodiments define a sequence of processing steps performed on the liquid immune cell cultures. Here, it becomes obvious, that different processing steps, comprising different unit operations, may be carried out, which provides flexibility.

According to various embodiments, the sequence of processing steps comprises at least one service operation to be performed at a service station. Such service operation is different from a unit operation and is not able to be performed at an above noted unit operation station. By separating service stations and unit operation stations, the steps to be performed may be divided between different stations, which adds flexibility in the design of the stations.

Various embodiments relate to a unit operation station of an integrated bioprocessing system for performing at least one bioprocess on liquid immune cell cultures.

Here, it can be that one of the units is a cartridge storage unit for receiving cartridges, which cartridges may each be preconfigured with a cartridge fluidic structure with respect to a predefined unit operation, that another of the units is a cartridge drive unit with a drive structure, that the unit operation station comprises a local transport mechanism and that in a standard routine, the local transport mechanism transports one of the preconfigured cartridges from a storage transfer location in the cartridge storage unit to a drive location of the cartridge drive unit, wherein the cartridge may be brought into an operative coupling with the cartridge drive unit to perform the respective unit operation.

The proposed unit operation station is part of a bioprocessing unit, which can serve for performing the above noted method. Therefore, all explanations given with regard to the above noted method are fully applicable.

With the cartridge fluidic structure according to various embodiments, a variety of unit operations may flexibly be realized, just by preconfiguration of the particular components. For the reception of those components, in various embodiments, a cartridge carrier is provided, which further can be part of the interface to the unit operation station. This way, the cartridge carrier provides the standardized part of the cartridge, while the cartridge fluidic structure provides the individualized part of the cartridge.

In various embodiments, the unit operation station provides at least one transfer location. In addition to the explanations given above, the transfer location can be a mechanical device designed for carrying the receptacles during performing the unit operations, which mechanical device can extend horizontally.

Various embodiments detail the interfaces provided by the drive structure. By providing different interfaces, the drive unit can be flexibly adapted to the cartridge fluidic structure of the cartridge.

According to various embodiments, the unit operation station is designed as a closed unit operation station, such that all liquids involved in performing the respective unit operation are being kept within a closed inner volume. In addition to the above noted explanations given with regard to the proposed method it may be pointed out, that this may be achieved by guiding the liquids through respective tubes.

Various embodiments detail the cartridge waste storage unit and transfer of cartridges from the drive location to the cartridge waste storage unit by the local transport mechanism. In addition to the explanations given above, it is of particular importance, that the local transport mechanism is adapted to transport the used cartridge from the drive location to the cartridge waste storage.

In order to support standardization of the unit operation station throughout the integrated bioprocessing system, it is advantageous, if the unit operation station comprises a station carrier. This ensures a predefined overall mechanical structure of the unit operation station. According to various embodiments, as a storage principle for the cartridge storage and/or the cartridge waste storage, vertically stacking the cartridges is proposed. With this, the cartridge fluidic structure may extend in the horizontal plane, which is flexible in view of the design of the cartridge fluidic structure. Also, such storage principle does not put high constructional demands on the local transport mechanism. This is particularly true if the storage principles of the cartridge storage and the cartridge waste storage are identical. Finally, vertically stacking the cartridges within the cartridge storage and/or the cartridge waste storage requires minimum storage space.

Various embodiments are directed to a transport movement of the cartridge from the cartridge storage unit to the drive location. This transport movement can include movements in two directions, which makes it possible to maximize the utilization of the existing workspace.

The temperature control of the cartridges according to various embodiments gives flexibility with regard to the point in time, at which the respective cartridge is being used for performing a unit operation. This flexibility is important in the above noted, case of performing various bioprocesses in parallel, which requires a certain amount of synchronization. The temperature control may be performed by an above noted electronic process control.

Various embodiments relate to an integrated bioprocessing system for performing a bioprocess on liquid immune cell cultures, wherein the bioprocess comprises a sequence of processing steps, wherein the sequence of processing steps comprises at least one unit operation, and that the integrated bioprocessing system comprises a number of above noted, proposed unit operation stations for performing the unit operations.

Here, it can be that each of the unit operation stations are being designed according to the above noted, second teaching.

As noted above, the proposed integrated bioprocessing system can serve for performing the above noted, proposed method. Therefore, all explanations given with regard to the above noted method are fully applicable. Also, the proposed integrated bioprocessing system comprises a number of above noted, proposed unit operation stations, such that again, all explanations given with regard to the unit operation station are fully applicable as well. It becomes apparent with this additional teaching, that the proposed method makes it possible to assemble the integrated bioprocessing system from standardized unit operation stations, which are adapted to perform unit operations based on the cartridges, which are stored in the respective cartridge storage unit.

According to various embodiments, at least two of the unit operation stations are identical to each other with the exception of the respective cartridge drive units. Here it is important that the unit operation stations are standardized as far as possible while still being flexibly adaptable to the respective unit operations to be performed. As noted above, this means that in some embodiments, the station carriers of at least two unit operation stations are identical to each other.

The same is to be said regarding the standardization of the cartridges according to some embodiments, as explained earlier.

Generally, the concept of the cartridges makes it possible, that one unit operation station may perform various different unit operations, which render the unit operation stations particularly flexible in their use. As another alternative, however, it may be provided, that all cartridges, that are stored in the cartridge storage of a unit operation station, are preconfigured to perform one and the same, predefined unit operation. Although reducing the above noted flexibility, this may simplify the overall process, as each unit operation station only performs one predefined unit operation.

Various embodiments are detailing the receptacles of the integrated bioprocessing system that are transported by a global transport mechanism. For a simple construction of the integrated bioprocessing system, it is of utmost advantage, if the receptacles are standardized. The transport of the receptacles is a key element for the successful automation of the bioprocesses, that are to be performed on the integrated bioprocessing system. A particularly simple approach is the design of the global transport mechanism comprising a conveyor system. This can be especially advantageous with the transfer locations being arranged in a first plane according to some embodiments.

In various embodiments, the global transport mechanism may well comprise a robotic system. Such a robotic system requires more constructional effort, but provides a higher degree of flexibility. In particular, the robotic system may be utilized for additional tasks such as the connection process explained earlier.

Various embodiments are directed to a structure of the receptacles, which supports an easy transport of the receptacle. In particular the variant of the receptacle comprising a tray with a basically flat receptacle carrier may easily be transported via an above noted conveyor system.

Various embodiments are directed to realizing a closed bioprocess including a number of closed unit operations, wherein all liquids involved in the bioprocess are being kept within a closed inner volume. Various embodiments support guiding the liquids through tubes, which may be connected and disconnected by a tube welding system in a connection process that is subject of some embodiments. In various embodiments, the global transport mechanism provides a connection handling mechanism. This double use of the global transport mechanism renders the integrated bioprocessing system particularly compact.

By using an above noted electronic process control to coordinate the execution of the bioprocesses and the unit operation stations, the integrated bioprocessing system may be flexibly adapted to numerous different bioprocesses comprising different unit operations and processing steps.

Various embodiments are directed to the above noted service operations, which are distinct from the unit operations. In order to support a mechanical structure, that may interact with the global transport system in a constructionally easy way, in some embodiments, that at least part of the service transfer locations are arranged in a second plane, which is offset from the first plane, such as with respect to a vertical direction.

The integrated bioprocessing system may comprise an enclosure, which houses at least part of the unit operation stations. With such an enclosure, predefined environmental conditions may be guaranteed. This may be especially advantageous during the transport of the receptacles by the global transport system. Process reproducibility may be largely increased by this measure. In various embodiments, the integrated bioprocessing system is a closed bioprocessing system irrespective of the enclosure. This means that in this variant, the enclosure does not need to contribute to the closed property of the integrated bioprocessing system.

Various embodiments provide a method for performing a bioprocess on liquid immune cell cultures comprising a sequence of processing steps, wherein the at least one bioprocess is being performed on an integrated bioprocessing system, wherein the sequence of processing steps comprises at least one unit operation, which is being performed within a unit operation station of the integrated bioprocessing system, wherein cartridges, that have been preconfigured with a cartridge fluidic structure with respect to at least one unit operation, are being stored in a cartridge storage unit of the unit operation station and that for performing a unit operation, in a standard routine, one of the cartridges is being transported from a storage transfer location in the cartridge storage unit of the unit operation station to a drive location of a cartridge drive unit of the unit operation station by a local transport mechanism of the unit operation station and is being brought into operative coupling with the cartridge drive unit.

In various embodiments, the at least one unit operation station comprises at least one transfer location, to which receptacles, that have been preconfigured with a receptacle fluidic structure for containing a liquid are being transported by a global transport mechanism of the integrated bioprocessing system, in order to transfer liquid between a cartridge, which has been transported to the drive location, and the receptacle.

In various embodiments, the integrated bioprocessing system comprises a number of unit operation stations.

In various embodiments, the at least one bioprocess is a closed bioprocess, such that all liquids involved in the respective unit operation are being kept within a closed inner volume. In various embodiments, for the transfer of liquid between the cartridge and the receptacle, a cartridge transfer tube of the cartridge and a receptacle transfer tube of the receptacle are being connected in a closed connection process by a tube connection system.

In various embodiments, at least two said bioprocesses are being performed at least partly simultaneously by the integrated bioprocessing system.

In various embodiments, the sequence of processing steps comprises at least one service operation, which is being performed within at least one service station of the integrated bioprocessing system, separately from the unit operation stations.

Various embodiments provide a unit operation station of an integrated bioprocessing system for performing at least one bioprocess on liquid immune cell cultures, wherein the sequence of processing steps comprises at least one unit operation, which is being performed within a unit operation station, wherein the unit operation station comprises a number of units, wherein one of the units is a cartridge storage unit for receiving cartridges, which cartridges may each be preconfigured with a cartridge fluidic structure with respect to at least one unit operation, that another one of the units is a cartridge drive unit with a drive structure, that the unit operation station comprises a local transport mechanism and that in a standard routine, the local transport mechanism may transport one of the preconfigured cartridges from a storage transfer location in the cartridge storage unit to a drive location of the cartridge drive unit, wherein the cartridge may be brought into an operative coupling with the cartridge drive unit to perform the respective unit operation.

In various embodiments, the cartridge fluidic structure comprises a cartridge liquid container such as a liquid bag, a functional device such as a separation device and a cartridge tube set for fluidically connecting those components to each other and/or to other components.

In various embodiments, the drive structure provides one or more of a mechanical interface, a fluidic interface and an electrical interface to the cartridge, which cartridge is located in the drive location.

In various embodiments, the transport of one of the cartridges from the cartridge storage unit to the cartridge drive unit involves one or more of a sideways movement of the respective cartridge out of the cartridge storage unit and an upward movement of the cartridge to position the cartridge in the drive location for a possible connection to a receptacle.

In various embodiments, the unit operation station comprises a temperature control device to control the temperature of the cartridges, which are being stored in the cartridge storage unit. Various embodiments provide an integrated bioprocessing system for performing a bioprocess on liquid immune cell cultures, wherein the bioprocess comprises a sequence of processing steps, wherein the sequence of processing steps comprises at least one unit operation, and that the integrated bioprocessing system comprises a number of unit operation stations for performing the unit operations, wherein each of the unit operation stations are being designed as described herein.

In various embodiments, at least two of the unit operation stations are identical to each other with the exception of the respective cartridge drive units, which may be preconfigured to the respective cartridges.

In various embodiments, the cartridges, that are being stored in a cartridge storage unit, are preconfigured such that the unit operation station is able to perform only one unit operation dedicated to this unit operation station, or, that the cartridges, that are being stored in a cartridge storage unit, are preconfigured such that the unit operation station is able to perform different unit operations depending on the configurations of the cartridges.

In various embodiments, the integrated bioprocessing system comprises a number of receptacles, that each have been preconfigured with a receptacle fluidic structure for containing a liquid and a global transport mechanism for the transport of the receptacles.

In various embodiments, the global transport mechanism comprises one of a conveyor mechanism, a belt conveyor mechanism, a roller conveyor mechanism, a ball conveyor system, and a robotic mechanism.

In various embodiments, the integrated bioprocessing system comprises a tube connection system which is designed to perform a closed connection process, connecting a cartridge transfer tube of a cartridge, located at a cartridge drive location, and a receptacle transfer tube of a receptacle, located at a transfer location.

In various embodiments, the integrated bioprocessing system comprises an electronic process control for coordinating the at least one bioprocess and that the electronic process control is designed to have at least two bioprocesses to be performed at least partly simultaneously, or to have at least two unit operation stations perform unit operations on different liquid immune cell cultures at least partly simultaneously.

In various embodiments, the integrated bioprocessing system comprises an enclosure, which houses at least part of the unit operation stations.

### BRIEF DESCRIPTION OF THE DRAWINGS

In the following, embodiments are explained with respect to the drawings. The drawing shows in

FIG. 1, a schematic representation of a proposed integrated bioprocessing system,

FIG. 2, in a perspective view a) a cartridge as such without the cartridge fluidic structure and b) in an explosive representation a cartridge with a cartridge fluidic structure and a unit operation station,

FIG. 3, a unit operation station of the bioprocessing system in FIG. 1 during performing a unit operation,

FIG. 4, the bioprocessing system of FIG. 1 a) in a sectional view along line IV-IV and b) in a sectional view along line V-V,

FIG. 5, a unit operation station of the bioprocessing system of FIG. 1 during the standard routine in a sequence a) to e),

FIG. 6, the unit operation station of FIG. 5 a) during a connecting process and b) during a disconnecting process and

FIG. 7, the unit operation station of FIG. 5 during the transport of a cartridge from the drive location to the cartridge waste storage in a sequence a) to b),

FIG. 8, the closed connection process in a schematic representation in a sequence a) to e).

### DETAILED DESCRIPTION

The integrated bioprocessing system 1 shown in the drawings can be adapted to perform a bioprocess for the manufacturing of genetically modified T cells. Here, T cells are genetically modified to express a chimeric antigen receptor (CAR). Consequently, the term “CAR-T cells” describes T cells that have been genetically modified to express a CAR. The genetically modified CAR-T cells, which represent the product of the bioprocess, may be administered to a patient and used to start or resume cancer treatment in the patient. As the bioprocess is performed, the initial immune cell culture is gradually processed. All explanations given are mainly directed to such a bioprocess. It may be pointed out, however, that those explanations are fully applicable to other bioprocesses as well.

The term “liquid immune cell culture” is to be understood in a broad sense and refers to an immune cell culture comprising at least one type of immune cells suspended as particles in any type of liquid. As will be explained below, the liquid immune cell culture may comprise other cell types that are not immune cells. Hence, the term “liquid immune cell culture” refers to a liquid immune cell culture at any stage during the bioprocess. Consequently, the type and fraction of immune cells present in the liquid immune cell culture will change during the bioprocess applied as certain immune cells are enriched or depleted from the liquid immune cell culture and/or the immune cells are genetically modified.

The term “immune cells” generally refers to different types of white blood cells. Hence, the term “immune cells” includes a variety of cells, for example, but not limited to dendritic cells, T lymphocytes, also referred to as T cells, B lymphocytes, natural killer cells, macrophages or the like. Immune cells may also include subtypes of immune cells, for example tumor-infiltrating lymphocytes or different types of T cells. Subtypes of a certain type of immune cells may be classified based on the type of antigen present at the cell surface. Hence, the term immune cells may for example refer to T cells comprising the surface antigen CD4 (“CD4+ T cells”). Typically, a certain type of immune cells, e.g., T cells, in some embodiments a certain subtype of immune cells, e.g., CD4+ T cells, will be selectively enriched by the bioprocess, while other immune cells, e.g. macrophages, and/or other cell types that are not immune cells, e.g., erythrocytes, and/or other subtypes of immune cells, e.g., CD8+ T cells, will be depleted from the liquid immune cell culture. The immune cells to be enriched are referred to as target immune cells, all other components to be depleted from the liquid immune cell culture are referred to “impurities”. Further, and as mentioned above, the target immune cells might be genetically modified.

The term “liquid” is to be understood in a broad sense as well and refers to any liquid and/or particle-containing liquid that is processed within the integrated bioprocessing system 1. Hence, the term liquid might refer to media, waste, the liquid immune cell culture, byproducts obtained during the bioprocess, samples and/or an initial immune cell culture.

The term “sample” refers to a smaller fraction of a liquid that has been separated from a larger portion of the liquid as part of a “sampling process” that will be explained later.

The term “media” refers to culture media, feed media, washing solutions, activation reagents, virus solutions containing virus particles and/or specific reagents required to perform at least a part of the bioprocess. The term “media” also includes particle-containing liquids such as magnetic beads suspended in a liquid.

The term “waste” refers to any liquid and/or particle-containing liquid obtained during the bioprocess, wherein the respective liquid and/or particle-containing liquid may be discarded and is not used further. Note that sampling may also be performed on waste prior to disposal. The term “initial immune cell culture” refers to a liquid immune cell culture before a first processing step of the bioprocess is applied. The initial immune cell culture might be derived from different sources. In an approach often referred to as “autologous cell therapy”, the initial immune cell culture is obtained from a donor, who is also the recipient of the product after the bioprocess has been performed completely. In “allogenic cell therapy”, the initial immune cell culture might be derived from at least one donor, who is not the recipient of the product. Additionally or alternatively, the initial immune cell culture might be derived from more than one donor and/or used for more than one recipient. In this case, the immune cell cultures obtained from different donors are combined into a single initial immune cell culture. In various embodiments, the initial immune cell culture is obtained in a process called “leukapharesis”. In leukapharesis, immune cells are obtained from the patient. Additionally or alternatively, the initial immune cell culture might also be obtained from a tissue of the patient. As mentioned above, the initial immune cell culture might also be obtained by one or more donors that are not the patient.

Depending on the source of the initial immune cell culture and the bioprocess to be carried out, the initial immune cell culture, particularly the type, amount and distribution of impurities as well as target immune cells might vary.

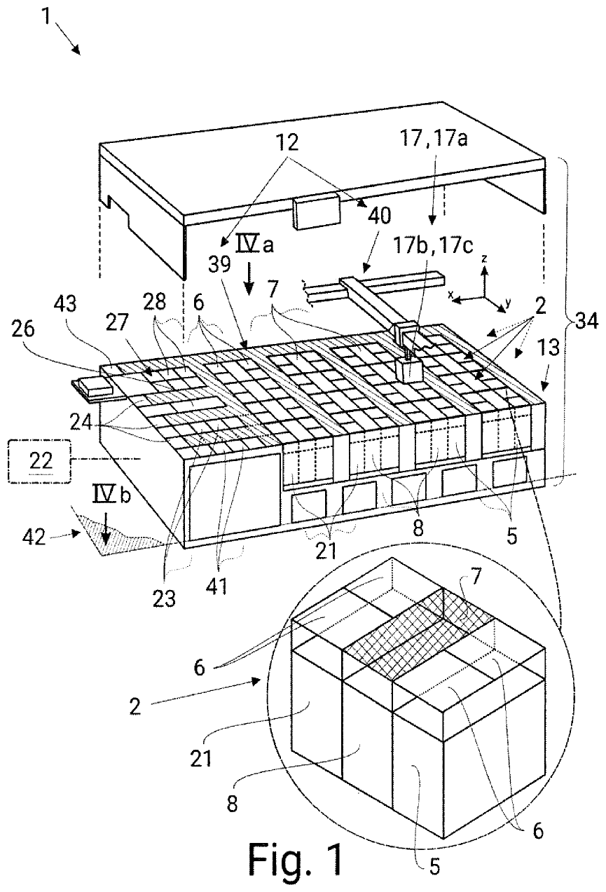

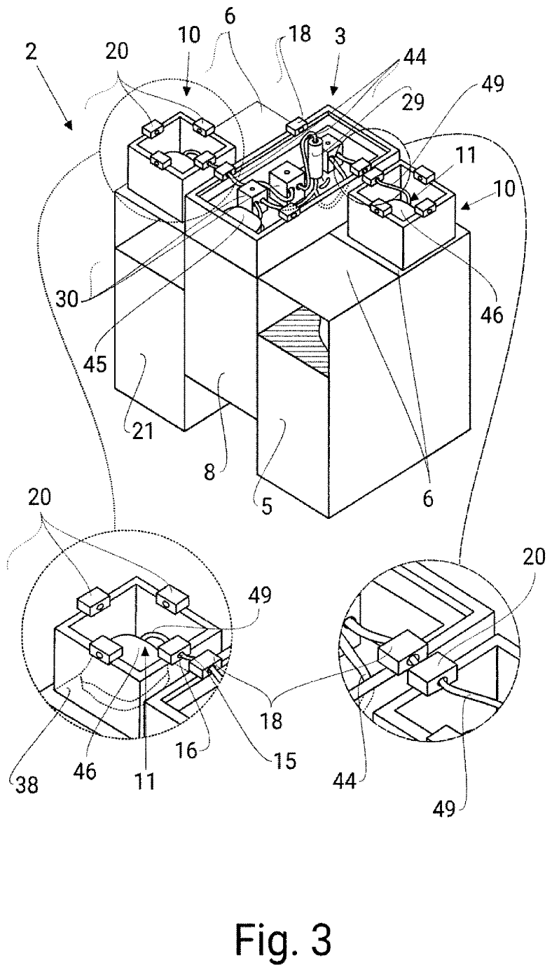

Proposed is a method for performing a bioprocess on liquid immune cell cultures comprising a sequence of processing steps wherein the at least one bioprocess is being performed on an integrated bioprocessing system 1 shown in FIG. 1. The sequence of processing steps comprises at least one unit operation, which is being performed within a unit operation station 2 of the integrated bioprocessing system 1. Such unit operation station 2 is shown in FIG. 3 during performing of the respective unit operation.

In various embodiments, the term “processing step” is to be understood in a broad sense and refers to a distinctive step that is being performed as part of a bioprocess involving a liquid immune cell culture. The type and sequence of processing steps performed depends on the bioprocess that is carried out on the respective liquid immune cell culture and on the type of (initial) immune cell culture. Depending on the mentioned parameters, different processing steps may be combined in any given sequence. Additionally or alternatively, the configuration of a processing step might differ, and/or processing steps may be repeated. Each processing step comprises at least one unit operation.

The term “unit operation” is an operation, that comprises at least one unit operation step or a sequence of unit operation steps. A unit operation is being performed at the respective unit operation station 2. The term “unit operation step” describes one step that is performed on the liquid immune cell culture at a respective unit operation station 2. The type, number and sequence of unit operation steps performed as part of a unit operation depend on the type of unit operation that is involved in a processing step and on the features of the liquid immune cell culture being processed. All unit operation steps of a unit operation are being performed within a unit operation station 2. In various embodiments, all the unit operation steps of a unit operation are being performed within one and the same unit operation station 2.

Essential is that cartridges 3 are provided, that may be preconfigured with a cartridge fluidic structure 4 with respect to at least one unit operation. Based on this, depending on the configuration of the cartridge fluidic structure 4, just about any unit operation may be realized with the preconfigured cartridge 3. This way, each cartridge fluidic structure 4 may be highly individualized for different bioprocesses and with that particularly for different liquid immune cell cultures as well.

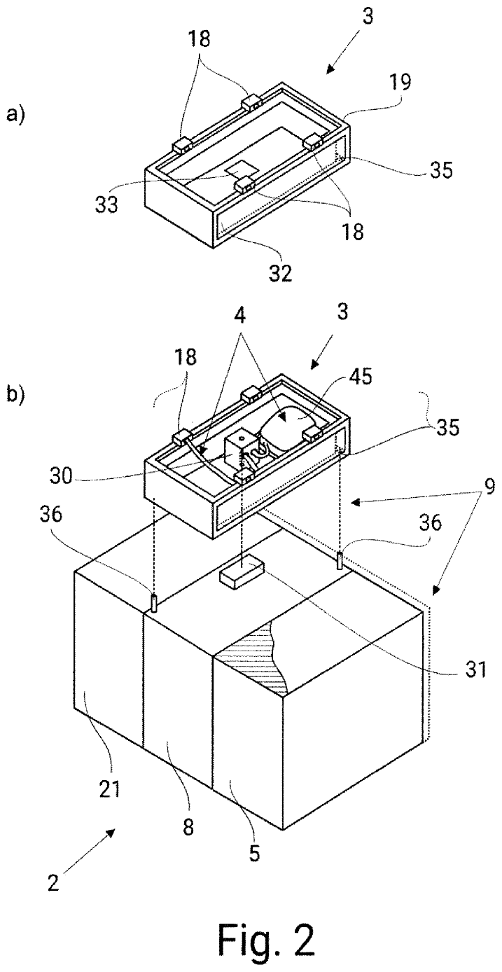

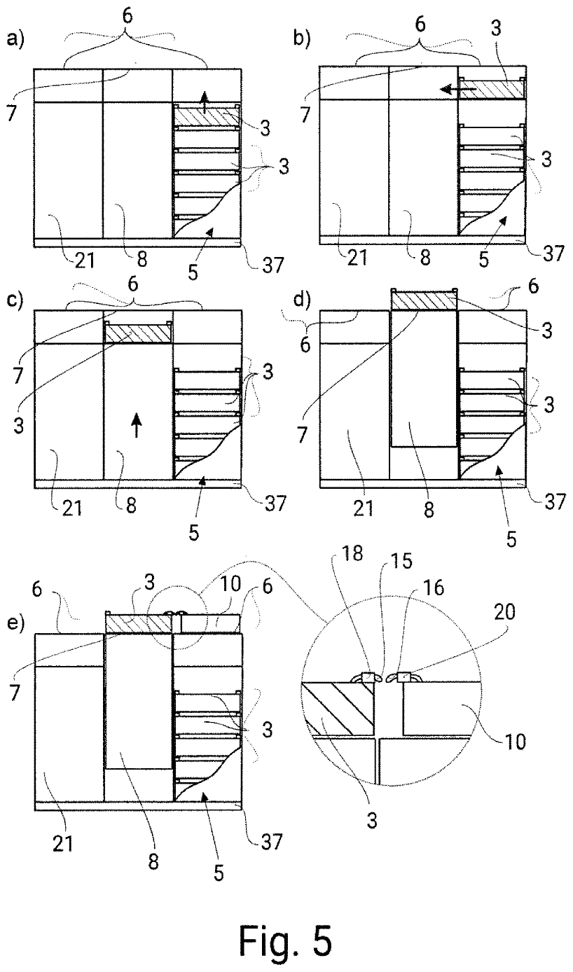

FIG. 2a shows a cartridge 3 in its not yet configured state without a cartridge fluidic structure 4, while FIG. 2b shows a cartridge 3, that has been preconfigured with a cartridge fluidic structure 4 with respect to at least one unit operation. Those preconfigured cartridges 3 are being stored in a cartridge storage unit 5 of the unit operation station 2, as is shown in FIG. 5, for example.

For performing a unit operation, a standard routine is defined. According to the standard routine, one of the cartridges 3 is being transported from the cartridge storage unit 5 of the unit operation station 2 to a drive location 7 of a cartridge drive unit 8 of the unit operation station 2 (sequence FIGS. 5a), 5b), 5c), 5d)) by a local transport mechanism 9 of the unit operation station 2, which is only indicated in FIG. 2b. Subsequently, the cartridge 3 is being brought into operative coupling with the cartridge drive unit 8.

In various embodiments, performing of each and any one of the unit operations includes performing the above noted standard routine, which provides the transport of the respective cartridge 3 from the cartridge storage unit 5 to the cartridge drive unit 8 and the operative coupling of the cartridge 3 with the cartridge drive unit 8. The subsequent performance of the unit operation is then individualized dependent from the preconfiguration of the cartridge 3.

In various embodiments, the standard routine is performed on all cartridges 3. However, in an alternative embodiment, in addition to the standard routine described above, a modified standard routine may be performed. In this modified standard routine, at least one of the cartridges 3 is transported to the drive location 7 by a global transport mechanism 12 to be explained in detail later. In the modified standard routine, the cartridges 3 may be stored in at least one global cartridge storage unit (not depicted) and retrieved from the global cartridge storage unit by the global transport mechanism 12. The global cartridge storage unit may comprise means to control, and in some embodiments also monitor, the temperature within the global cartridge storage unit. Here, the same means as will be described for the cartridge storage unit 5 below, may be used. Here it is also possible to have more than one global cartridge storage unit so that the cartridges 3 may be stored according to the storage temperatures required.

The operative coupling between the cartridge 3 and the cartridge drive unit 8 makes it possible for the cartridge 3 to be a completely passive component, without any kind of actuators. Via this operative coupling, any actuation may be transmitted from the cartridge drive unit 8 to the cartridge 3. However, it is generally possible, that the cartridge 3 comprises additional actuators.

According to various embodiments it is proposed, that at least two of the cartridges 3, such as all cartridges 3, are identical to each other with the exception of the respective cartridge fluidic structures 4, which may be configured to the respective unit operations. This means, that as far as the respective cartridge fluidic structure 4 is concerned, the cartridges 3 may (but do not have to) deviate from each other. With the identical cartridges 3 in this sense, not only the transport of the cartridges 3 may be standardized, but also the transfer of liquids to and from the cartridges 3.

According to another embodiment it is proposed, that the at least one unit operation station 2 comprises at least one transfer location 6, indicated in FIG. 3. In various embodiments, the transfer location 6 is provided by a surface or a pad for receiving a receptacle 10. Accordingly, receptacles 10, that have been preconfigured with a receptacle fluidic structure 11 for containing a liquid, such as an immune cell culture, are being transported by a global transport mechanism 12, depicted in FIG. 1, to a transfer location 6, in order to transfer liquid between a cartridge 3, which has been transported to the drive location 7, and the receptacle 10 and/or to perform a respective unit operation on the liquid contained in the receptacle 10. In various embodiments, each unit operation station 2 comprises four transfer locations 6. As shown in FIG. 1 and in an insofar various embodiments, each transfer location 6 is of a rectangular geometry, with two transfer locations 6 being located next to each other. In various embodiments, two pairs of transfer locations 6 are located opposite each other with the drive location 7, which will be explained later, located between each pair of transfer locations 6.

As will be explained below, the receptacles 10 located in the transfer locations 6 need to be aligned to the cartridge 3 located in the drive location 7. For alignment, either the global transport mechanism 12, in some embodiments a robotic mechanism 40 detailed further below, may be used or the transfer location 6 may comprise positioning means to align the receptacle 10 to the cartridge 3.

According to one embodiment it is proposed, that at least two of the receptacles 10, in some embodiments all receptacles 10, are identical to each other with the exception of the respective receptacle fluidic structures 11, which may be configured to the respective liquid to be contained. This means, that as far as the respective receptacle fluidic structure 11 is concerned, the receptacles 10 may (but do not have to) deviate from each other. The liquid to be contained in the receptacle fluidic structure 11 may depend on the process step being performed. This means, that the complete handling of the receptacles 10 including transport, establishing a fluid connection etc. may be standardized for all receptacles 10. An example for those identical receptacles 10 is shown in FIG. 3.

According to one embodiment it is proposed, that the integrated bioprocessing system 1 comprises a number of unit operation stations 2, in some embodiments, that the transfer locations 6 of the unit operation stations 2 are arranged in a first plane 13 and that the receptacles 10 are being transported by the global transport mechanism 12 to and from the transfer locations 6 in this first plane 13. The first plane 13 can be aligned horizontally. By arranging the transfer locations 6 in the first plane 13, transport of the receptacles 10 to and from the transfer locations 6 is simplified. In addition, and as will be explained below, the receptacles 10 are then located in the same plane as the cartridge 3 that is located in the drive location 7.

In various embodiments, at least part of the number of unit operation stations 2 are arranged in unit operation station slices 14. Each unit operation station slice 14 can include separate transport devices such as wheels or the like to move the unit operation station slices 14 into and out of the integrated bioprocessing system 1. By being able to remove a unit operation station slice 14 including the number of unit operation stations 2 located within that unit operation station slice 14 from the integrated bioprocessing system 1, servicing of the unit operation stations 2, which will be explained further below, is particularly simple. Further, a unit operation station slice 14 may be removed from or introduced into the integrated bioprocessing system 1 without affecting the operability of the remaining unit operation stations 2 within the integrated bioprocessing system 1. This is particularly advantageous, if a part of the integrated bioprocessing system 1 needs to be accessed for maintenance.

Each unit operation station slice 14 can contain at least two, or at least four unit operation stations 2. In various embodiments, the integrated bioprocessing system 1 comprises at least one, at least two, or at least four unit operation station slices 14, in some embodiments arranged next to each other within the integrated bioprocessing system 1. By arranging the unit operation stations 2 in unit operation station slices 14, a number of unit operation stations 2 may be moved at once. This way, the exchange of a number of unit operation stations 2 is particularly simple.

According to one embodiment it is proposed, that at least two of the unit operation stations 2 are identical to each other with the exception of the respective cartridge drive units 8, which may be preconfigured to the respective cartridges 3. This may be taken from FIG. 1, for example. This leads to another degree of standardization, which allows for scaling the integrated bioprocessing system 1 up simply by adding unit operation stations 2, which are basically identical to each other in a way noted above and with the exception of the cartridge fluidic structure 4 as also noted above.

According to one embodiment it is proposed, that the at least one bioprocess is a closed bioprocess, such that all liquids involved in the respective unit operation are being kept within a closed inner volume. In various embodiments, this closed structure is realized by performing at least part of the liquid handling involved in the respective unit operation within tubes. This renders the arrangement of the transfer locations 6 in a first plane 13 very effective, as a tubular connection for the transfer of liquid may easily be established in such a first plane 13.

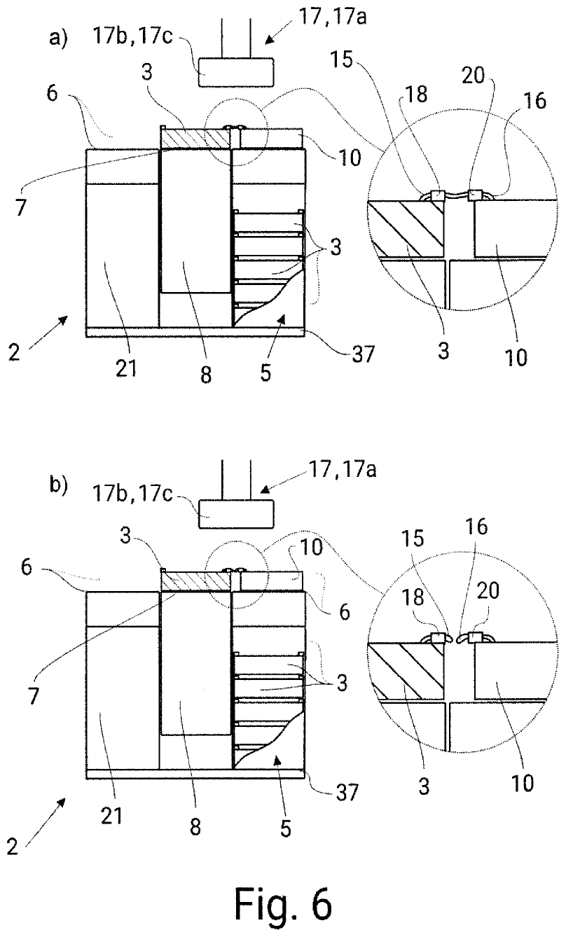

For an easy realization of a closed bioprocess it is proposed, that for the transfer of liquid between the cartridge 3 and the receptacle 10, a cartridge transfer tube 15 of the cartridge 3 and a receptacle transfer tube 16 of the receptacle 10 are being connected in a closed connection process by a tube connection system 17. This is indicated in FIG. 6a. In various embodiments, the connection process is being performed by a tube welding system 17a.

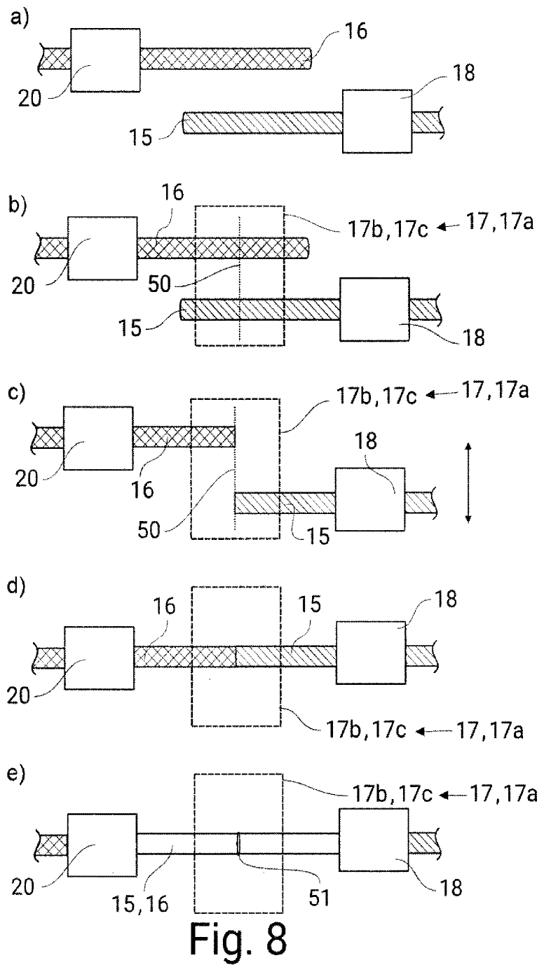

The connection process is shown in FIG. 8 and will be described in detail later. The connection process is performed by the tube connection system 17, in particular by a tube handling device (not shown) of the tube connection system 17. It can also comprises a step of arranging the tubes 15, 16 to be connected relative to the rest of the tube connection system 17 (sequence FIG. 8a) to FIG. 8b)) and a subsequent step of welding the tubes 15, 16 to be connected by the tube connection system 17 (FIG. 8d)). In various embodiments, before the step of welding, a step of trimming the tubes 15, 16 to be connected is provided within the connection process (FIG. 8b)).

As depicted in FIG. 3, the cartridge transfer tube 15 and the receptacle transfer tube 16 are provided by the cartridge fluidic structure 4 and the receptacle fluidic structure 11. In various embodiments, the cartridge fluidic structure 4 and the receptacle fluidic structure 11 have been preassembled in such a way that each cartridge transfer tube 15 is located in a predefined cartridge anchor point 18 provided by the cartridge frame 19 and that each receptacle transfer tube 16 is located in a predefined receptacle anchor point 20 provided by the receptacle 10. This is shown in FIG. 3, for example. In various embodiments, the cartridge anchor points 18 and the receptacle anchor points 20 are arranged in such a way that one cartridge anchor point 18 faces one receptacle anchor point 20, when the respective cartridge 3 is in the drive location 7 and the respective receptacle 10 is in the transfer location 6.

In various embodiments, and as depicted in FIG. 3, the cartridge transfer tube 15 protrudes out of the cartridge anchor point 18 towards the transfer location 6 that the receptacle 10 to be connected is located in. It can also be that the receptacle transfer tube 16 protrudes out of the receptacle anchor point 20 towards the cartridge anchor point 18 that the cartridge transfer tube 15 is located in. A welding system 17a, which will be described in more detail below, connects the receptacle transfer tube 16 and the cartridge transfer tube 15 in the closed connection process.

In addition, as indicated in FIG. 6b, the welding system 17a can be designed to perform a disconnecting process as well. In various embodiments, the transfer tubes to be disconnected are closed and cut at their respective ends, such that the inner volumes of the tubes remain sterile. Here it may also be pointed out, that the use of such a welding system 17a enables to perform multiple welds subsequently on the same transfer tube. This provides flexibility as for example the same receptacle transfer tube 16 may be subsequently connected to different cartridge transfer tubes 15.

According to one embodiment it is proposed, that the at least one unit operation station 2 comprises a cartridge waste storage unit 21 for receiving the cartridges 3 for their later disposal and that after performing the unit operation, the cartridge 3 is being transported to the cartridge waste storage unit 21 by the local transport mechanism 9. By using a cartridge waste storage unit 21 to receive the cartridges 3 after the unit operation has been performed, the cartridges 3 may be disposed locally without the need to utilize the global transport mechanism 12. This way, disposal is simplified. In addition, the cartridge waste storage unit 21 only needs to be retrieved periodically. As will be explained later, it can be that the cartridges 3 are stacked in a vertical orientation within the cartridge waste storage unit 21. While the transport of the cartridges 3 to the cartridge waste storage unit 21 is provided in some embodiments, it is also possible that the cartridges 3 are transported to a global cartridge waste storage unit (not depicted). In various embodiments, the cartridges 3 are transported to the global cartridge waste storage unit by the global transport mechanism 12. Here it should be noted that cartridges 3 that were used at different unit operation stations 2 may be transported to the same global cartridge waste storage unit.

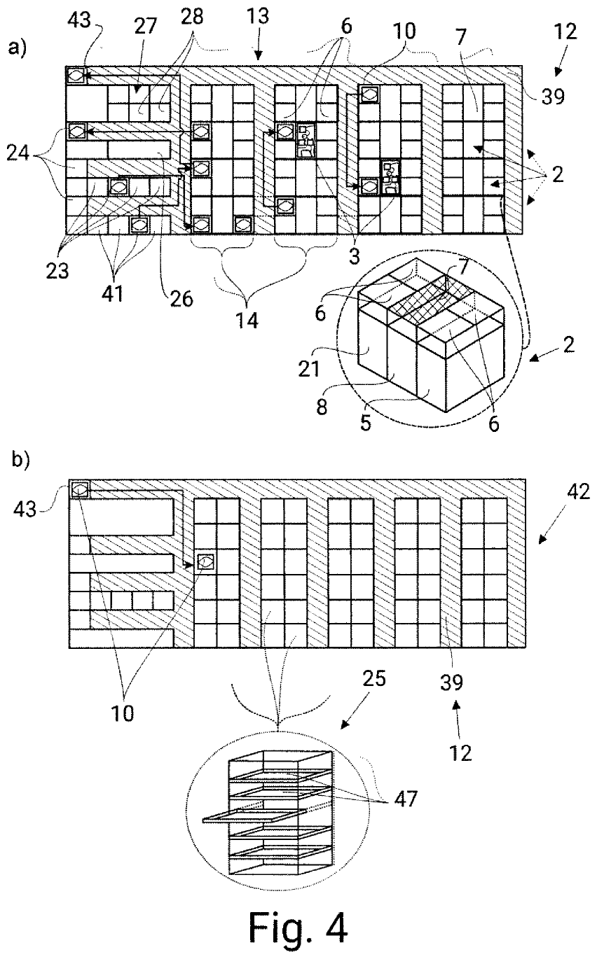

According to one embodiment it is proposed, that at least two said bioprocesses are being performed at least partly simultaneously by the integrated bioprocessing system 1, in some embodiments, as coordinated by an electronic process control 22. This is indicated in FIG. 4a with two cartridges 3 of two separate unit operation stations 2 having been transported to a respective drive location 7. An interesting aspect here is that the integrated bioprocessing system 1 may easily be adapted in size. For process development, where all unit operation stations 2 may be configured to perform only one and particularly also the same unit operation, it can be that up to 5 bioprocesses are performed at least partly simultaneously. In a small-scale manufacturing plant, the number of bioprocesses to be performed at least partly simultaneously is envisaged to be around 20. In a large-scale manufacturing plant, the number of bioprocesses to be performed at least partly simultaneously is around 50. However, it should be noted, that the scalability of the bioprocessing system 1 and the number of bioprocesses that are performed at least partly simultaneously is not limited.

By performing at least two bioprocesses at least partly simultaneously, the throughput is enhanced. Here the full flexibility of the integrated bioprocessing system 1 regarding the layout of the unit operation stations 2, service stations, central supply storages 23 and central waste storages 24 is particularly advantageous, because at least part of the mentioned elements may be adapted to the type and number of bioprocesses to be performed.

The electronic process control 22 shown in FIG. 1 is designed to coordinate the bioprocesses performed in parallel, such that it is ensured that each bioprocess is carried out according to its specific protocol. For this, the electronic process control 22 coordinates the utilization of all elements required for each bioprocess and between the at least two bioprocesses. For example, if two bioprocesses require a processing step “enrichment”, the electronic process control 22 may decide, which particular unit operation station 2 is used to carry out the unit operation configured for the processing step enrichment for each bioprocess. If for example, four unit operation stations 2 are available for “enrichment”, the electronic process control 22 may assign a first unit operation station 2 to the first bioprocess and a second unit operation station 2 to the second bioprocess.

The above noted electronic process control 22 in the shown embodiment is realized as one central electronic process control. However, the electronic process control 22 may well be realized at least partly in a decentralized structure. For example, the unit operation stations 2 may each comprise a local electronic process control, wherein the local electronic process controls are coordinating themselves are being coordinated by a global electronic process control.

In any case, for performing at least two bioprocesses simultaneously, the coordination by the electronic process control 22 may follow an optimization strategy in view of cost efficiency, time efficiency, quality or the like. Those criteria can then be time efficiency, quality, cost efficiency or the like, as noted above.

According to one embodiment it is proposed, that the sequence of processing steps performed on the liquid immune cell cultures comprises at least one processing step of the group of enrichment, selection, activation, modification (includes expansion) and formulation (includes fill), which processing step comprises at least one unit operation which unit operation comprises at least one unit operation step. Here it becomes clear, that it considerably enhances process flexibility, to store cartridges 3 for different unit operations in one and the same unit operation station 2. With this it is generally possible to perform two or more processing steps, that comprise a number of different unit operations, in one and the same unit operation station 2. Additionally or alternatively, one unit operation station 2 may be configured to perform one and the same unit operation.

In various embodiments, the enrichment processing step at least comprises a counter-flow centrifugation unit operation step or an acoustic separation unit operation step. By using counter-flow centrifugation, the target immune cells within the liquid immune cell culture are separated from other (immune) cells and impurities present within the liquid immune cell culture based on different cell sizes. In various embodiments, the target immune cells will be separated from other immune cells and impurities by acoustic separation. Here, acoustic waves are used to separate different cell types and/or impurities based on size, compressibility, and mechanical properties.

According to various embodiments it is proposed, that the selection processing step at least comprises a magnetic separation unit operation step. By using magnetic separation, the target immune cells, such as a specific subtype of target immune cells, will be selectively enriched using magnetic particles. Selective enrichment might be achieved by selectively binding the specific subtype of immune cells to magnetic particles. For this, the magnetic particles are coated with antibodies that are directed to antigens that are unique to the immune cell of interest.

According to various embodiments it is proposed, that the activation processing step at least comprises a reagent addition unit operation step and an incubation step. Addition of a reagent is used to activate the target immune cells. For activation, an activation reagent is added to the liquid immune cell culture. As will be explained in detail below when an example for a method for operating a bioprocess is given in more detail, an incubation step is carried out during the activation processing step. Finally, a washing unit operation step is performed. As will be explained below, the washing unit operation step may also be carried out as part of the counter-flow unit operation step. Alternatively, the washing unit operation step may also be performed using acoustic technology.

After activation, genetic modification of the target immune cells may be performed. In the modification processing step, genetic modification of the liquid immune cell culture is performed. In various embodiments, and as will be described in further detail below, genetic modification is performed by viral vector addition. It can be that the viral vector comprises a transgene. Additionally, and to enhance the efficiency of the modification step, certain reagents may also be added to the liquid immune cell culture. Additionally, a centrifugation unit operation step, e.g., by counter-flow centrifugation, may be carried out to improve the colocalization of the viral vector and the target immune cells.

To perform the modification processing step, at least a unit operation step of viral vector addition is performed. It can be that the modification processing step comprises an expansion step that is performed at an incubator service station 25, again to be explained below. However, it should be noted here, that the expansion step might also be omitted, if it is not required in the bioprocess to be performed.

As mentioned above, in various embodiments, to facilitate the uptake of the viral vector by the target immune cells within the liquid immune cell culture, viral transduction is used. Alternatively, electroporation may be used.

According to various embodiments it is proposed, that the formulation processing step at least comprises an acoustic separation unit operation step or a counter-flow centrifugation unit operation step to concentrate the target immune cells.

It is to be noted, that there are several additional process steps possible, that are not performed at the unit operation stations. Accordingly it can be that the sequence of processing steps comprises at least one service operation, which is being performed within at least one service station of the integrated bioprocessing system 1, separately from the unit operation stations 2. In various embodiments, the at least one service operation may comprise an incubation step, a sampling step, a media fill step, or the like.

By having dedicated service stations to perform an incubation step, a sampling step or a media fill step, steps that are independent of the unit operation to be performed within the unit operation station, are conducted at the respective service stations. For example, the incubation step is performed within an incubator service station 25. It can be that the sampling step is performed within a sampling service station 26. It can be that the media fill step is performed within a media fill service station 27. The respective service stations will be described in more detail below.

In various embodiments, the media fill step is performed at a media fill service station 27 comprising a number of media fill locations 28 for the placement of receptacles 10. In various embodiments, the media fill service station 27 comprises a number of media storage containers providing at least one type of media. Each media storage container comprises at least one media container transfer tube. Each media storage container may be equipped with means for stirring the liquid contained within the media storage container. Additionally or alternatively, each media storage container may be equipped with means to adjust the temperature of the liquid within the media storage container. Such means may be a temperature control element provided below the media storage container. Further the media storage container may be provided with means to record the temperature of the liquid within the media storage container. For example, the media storage container may comprise a temperature probe. Additionally or alternatively, the media storage container may comprise means to determine the mass of the media storage container. This may be realized by placing the media storage container on a balance and/or load cells may be used. From the determined mass, a liquid filling level within the media storage container may be determined.

Examples for media located at a media fill service station 27 include culture media required in an expansion step. Additionally or alternatively, liquids that may for a number of reasons not be stored within the cartridge storage unit 5 of the unit operation station 2, may be provided by the media fill service station 27. This includes for example liquids that cannot be stored for a predefined period of time at the conditions provided by the cartridge storage unit 5 or the like.

The media fill service station 27 further comprises at least one liquid transfer element to transfer a liquid from the media storage container. The liquid transfer element can be a peristaltic pump 30. It can be that the media container transfer tube is inserted in the peristaltic pump 30 head so that liquid may be transferred from the media storage container. Alternatively, also means like vacuum pumps for generating an underpressure within the media container transfer tube may be used for liquid transfer.

In various embodiments, the liquid contained in the media storage container is transferred from the media storage container to a receptacle 10 comprising a receptacle fluidic structure 11 that does not contain a liquid within its liquid container, yet. The respective receptacle 10 comprising the receptacle fluidic structure 11 may be supplied from one of the central supply storages 23 and transferred to a media fill location 28 of the media fill service station 27 by the global transport mechanism 12. A flow sensor may be located at the media container transfer tube to measure the amount of liquid being transferred from the media storage container to the liquid container of the receptacle 10 and/or control the liquid transfer element in such a way, that a predefined amount of liquid is transferred to the liquid container of the receptacle 10. Additionally or alternatively, the means to determine the mass of the media storage container described above may be used to measure and/or control the transfer of liquid from the media storage container to the liquid container of the receptacle 10.

The fluidic connection between the media storage container and the receptacle 10 can be established by the welding system 17a that is also used in the closed connection process between the cartridge 3 and the receptacle 10. Hence, the transfer of liquids from the media storage container to a receptacle 10 can be a closed connection process as noted above.

Another teaching which is of equal importance relates to a unit operation station 2 as such of an integrated bioprocessing system 1 for performing at least one bioprocess on liquid immune cell cultures, wherein the sequence of processing steps comprises at least one unit operation, which is being performed within a unit operation station 2, wherein the unit operation station 2 comprises a number of units.

It is essential for this teaching, that one of the units is a cartridge storage unit 5 for receiving cartridges 3, which cartridges 3 may each be preconfigured with a cartridge fluidic structure 4 with respect to at least one unit operation, that another one of the units is a cartridge drive unit 8 with a drive structure 31, that the unit operation station 2 comprises a local transport mechanism 9 and that in a standard routine, the local transport mechanism 9 may transport one of the preconfigured cartridges 3 from a storage transfer location in the cartridge storage unit 5 to a drive location 7 of the cartridge drive unit 8, wherein the cartridge 3 may be brought into an operative coupling with the cartridge drive unit 8 to perform the respective unit operation.

The proposed unit operation station 2 can server to perform an above noted method according to the first teaching. All explanations with regard to the first teaching are equally applicable. According to one embodiment it is proposed, that the cartridge fluidic structure 4 comprises a cartridge liquid container 45 such as a liquid bag, a functional device 29 such as a separation device and a cartridge tube set 44 for fluidically connecting those components to each other and/or to other components. This is indicated in FIG. 2b. The functional device 29 serves to perform at least one unit operation step of the respective unit operation. Depending on the processing step and unit operation to be performed, the functional device 29 may have different functions such as an above noted separation function to separate different cell types within the liquid immune cell culture. It is to be noted that the cartridge 3 comprises any number of functional devices 29 that are required by the respective unit operation and the unit operation steps assigned to the unit operation. In various embodiments, at least part of the components of the cartridge fluidic structure 4 are composed of a single-use material. It can be that at least all components of the cartridge fluidic structure 4 that are configured to come into contact with a liquid are composed of a single-use material.

In various embodiments, the cartridge fluidic structure 4 provides at least one functional device 29 in the form of a liquid transfer element to transfer a liquid within the cartridge 3 and/or from the cartridge 3 to the receptacle 10 and/or from the receptacle 10 to the cartridge 3. In various embodiments, the liquid transfer element is a positive displacement pump. It can be that the liquid transfer element is a peristaltic pump 30. Additionally or alternatively, liquid transfer may be realized by generating an underpressure within the cartridge fluidic structure 4. The peristaltic pump 30 of the cartridge fluidic structure 4 can be actuated by the drive structure 31 of the respective cartridge drive unit 8. For this, during the standard routine, the cartridge fluidic structure 4 and with it the peristaltic pump 30 is being brought into operative coupling with the cartridge drive unit 8 and with it the drive structure 31. In addition, at least one flow sensor may be located at the cartridge tube set 44 to measure the amount of liquid being transferred within the cartridge tube set 44 and/or to control the liquid transfer element in such a way, that a predefined amount of liquid is transferred within the cartridge 3 and/or from the cartridge 3 to the receptacle 10 and/or from the receptacle 10 to the cartridge 3. The flow sensor of the cartridge fluidic structure 4 can be actuated, specifically powered, by the drive structure 31 of the respective drive unit 8. For this, an electronic sensor interface is provided by the drive structure 31 of the cartridge drive unit 8. Again and as already explained for the liquid transfer element above, during the standard routine, the cartridge fluidic structure 4 and with it the flow sensor is being brought into operative coupling with the cartridge drive unit 8 and with it the drive structure 31.

In this shown embodiment, the cartridge 3 comprises a cartridge carrier 32, which receives the components of the cartridge fluidic structure 4. The advantage is that the cartridge carrier 32 provides a standardized structure to receive the cartridge fluidic structure 4. Hence, the size of the cartridge carriers 32 can be identical for all unit operation stations 2 and, thus, independent of the specific unit operation performed at a unit operation station 2. Accordingly, the cartridge fluidic structure 4 can be arranged as desired within the cartridge carrier 32 while the cartridge carrier 32 provides a standardized interface to the cartridge frame 19 and to the cartridge drive unit 8. Additionally, the cartridge carrier 32 is out of contact with any liquids handled at the unit operation station 2. Hence, the cartridge carrier 32 can be reused after the cartridge 3 has been used in a unit operation. Further, using a cartridge carrier 32 to receive the components of the cartridge fluidic structure 4 ensures that any liquid that may leak from the cartridge fluidic structure 4, for example in case of a breach in the cartridge liquid container 45, is contained within the cartridge 3 and does not contaminate other elements, for example the cartridge drive unit 8 of the integrated bioprocessing system 1.

As depicted in FIG. 2b, the cartridge carrier 32 may comprise drive recesses 33 to enable the drive structure 31 of the cartridge drive unit 8 to engage with components of the cartridge fluidic structure 4. For example, and as depicted in FIG. 2a, the cartridge carrier 32 may comprise a drive recess 33 at a position, a pump head of a peristaltic pump 30 is to be placed. For actuation of the components of the cartridge fluidic structure 4 by the interfaces provided by the drive structure 31, it is important that the drive recesses 33 in the cartridge carrier 32 are aligned to the respective interfaces of the drive structure 31.

In various embodiments, the material of the cartridge carrier 32 is a multi-use material like plastic or the like. Further, and as mentioned above, each cartridge carrier 32 is standardized regarding its size. It can also be that the dimensions of the cartridge carrier 32 regarding its longitudinal and transverse direction correspond to the cartridge drive unit 8. As will be explained below, the cartridge drive structure 31 of the cartridge drive unit 8 provides interfaces that engage with the components of the cartridge fluidic structure 4.

The cartridge carrier 32 can be enclosed by a cartridge frame 19 as depicted in FIG. 2a. The cartridge frame 19 provides a rigid structure for the cartridge carrier 32. In various embodiments, the cartridge frame 19 is also composed of a multi-use material like plastic or the like.

Here it is important that each cartridge carrier 32 may be combined with each cartridge frame 19. This simplifies the preassembly and disassembly of the cartridges 3. The disassembly and reassembly are particularly simple, as only the used cartridge fluidic structure 4 needs to be removed from the cartridge 3 and exchanged by a new cartridge fluidic structure 4. This way, after the unit operation station 2 has been removed from the enclosure 34, servicing of the unit operation station 2 is particularly simple as the cartridge carrier 32 and the cartridge frame 19 may be reused directly and moved from a cartridge waste storage unit 21 to the cartridge storage unit 5. Here it should also be noted, that arranging at least part of the unit operation stations 2 on the at least one unit operation station slice 14, that was explained above, further contributes to a simplified servicing of the unit operation stations 2 as multiple unit operation stations 2 may be removed from the integrated bioprocessing system 1 at the same time.

The cartridge 3 and the cartridge drive unit 8 are aligned as part of the operative coupling that was mentioned above. To align the cartridge 3 and the cartridge drive unit 8, the cartridge frame 19 may comprise a number of alignment recesses 35. In various embodiments, the alignment recesses 35 have a round geometry and align with the alignment elements 36 located on the cartridge drive unit 8.

The cartridge frame 19 comprises a number of above noted cartridge anchor points 18. In various embodiments, the cartridge transfer tubes 15 are placed in the cartridge anchor points 18 during the preassembly process of the cartridges 3.

According to various embodiments it is proposed, that the unit operation station 2 comprises at least one above noted transfer location 6, on which a receptacle 10, that has been preconfigured with a receptacle fluidic structure 11 for containing a liquid, such as an immune cell culture, may be placed to transfer liquid between a cartridge 3, which is located at the drive location 7, and the receptacle 10.

An embodiment for the unit operation station 2 is shown in FIG. 3. According to this embodiment, each transfer location 6 of the unit operation station 2 is provided by the top of a unit other than the cartridge drive unit 8. As may be taken from FIG. 3, this allows to fluidically connect a receptacle 10, which has been transported to one of the transfer locations 6, to the cartridge 3, which has been transported to the drive location 7.

According to one embodiment it is proposed, that the drive structure 31 provides a mechanical interface, such as a mechanical coupling for the rotational drive of a centrifuge or a pump. This may regard a peristaltic pump 30 noted above, for example. The drive structure 31 may also provide a fluidic interface, such as a vacuum connection to generate an underpressure in the cartridge fluidic structure 4, and/or an electrical interface, such as an electronic sensor interface, to the cartridge 3, which cartridge 3 is located in the drive location 7. Accordingly, it can be that the interfaces of the drive structure 31 are designed to be used repeatedly with different cartridges 3. By using the drive structure 31 to provide different interfaces for the cartridge fluidic structure 4, the drive structure 31 may be customized to the respective cartridge fluidic structure 4. Further, the different unit operations may be performed at a unit operation station 2 by exchanging the cartridge fluidic structure 4 and/or the drive structure 31.

It is to be noted that the drive structure 31 may be tailored to the respective unit operation to be performed. However, it is also well possible to use a standardized drive structure 31, and consequently and as mentioned above, a standardized cartridge carrier 32, for at least part of the different unit operation stations 2. This makes the exchange of the unit operation stations 2 even more simple.

According to one embodiment it is proposed, that the unit operation station 2 is designed as a closed unit operation station 2 to perform closed unit operations, such that all liquids involved in performing the respective unit operation are being kept within a closed inner volume, in some embodiments, that at least part of the liquid handling involved in the respective unit operation is being performed within tubes. A closed, above noted bioprocess may easily be realized utilizing such closed unit operation stations 2.

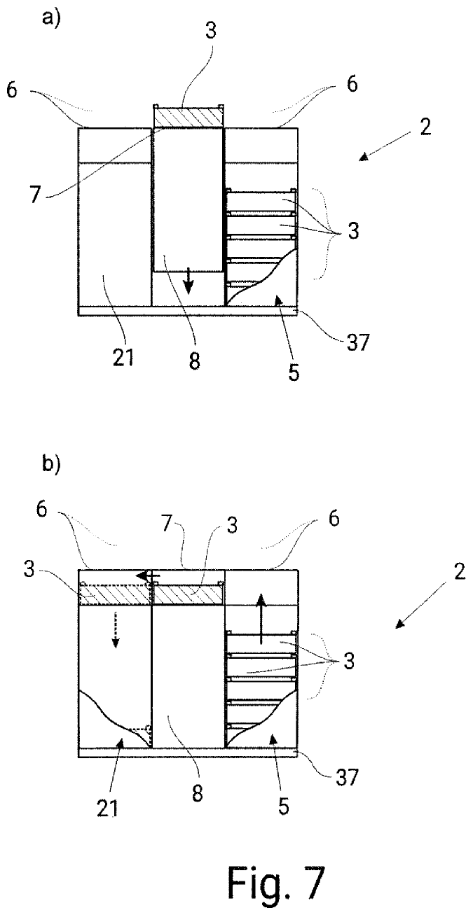

As shown in FIG. 7 it is proposed, that another unit of the unit operation station 2 is an above noted cartridge waste storage unit 21 for receiving the cartridges 3 for their later disposal. The local transport mechanism 9 can be designed to transport a cartridge 3 to the cartridge waste storage unit 21, such as from the drive location 7 as shown in FIG. 7.

According to one embodiment it is proposed, that the unit operation station 2 comprises a station carrier 37, which receives all of said units of the unit operation station 2. The station carrier 37 is indicated in FIGS. 5 to 7.

With the station carrier 37, all units of the unit operation station 2, here the cartridge storage unit 5, the cartridge drive unit 8 and the cartridge waste storage unit 21, are combined to one compact entity, that may be handled and transported as such. For example, a unit operation station 2 may easily be exchanged by another unit operation station 2 in case of failure of one of the units, for example of the cartridge drive unit 8. This simple option of exchanging a unit operation station 2 also contributes to the process robustness. For a compact arrangement, the units of the unit operation station 2 are arranged side by side.

According to one embodiment it is proposed, that the cartridge storage unit 5 and/or the cartridge waste storage unit 21 receives the cartridges 3 in a vertically stacked manner. This is shown in FIGS. 5 to 7 as well. In the shown embodiment, the cartridge storage unit 5 and/or the cartridge waste storage unit 21 is structured as a first-in-last out storage. Alternatively, the cartridge storage unit 5 and/or the cartridge waste storage unit 21 is structured as a first-in-first-out storage.

The sequence of FIGS. 5a), 5b) and 5c) shows, that the transport of one of the cartridges 3 from the cartridge storage unit 5 to the cartridge drive unit 8 involves a sideways, such as horizontal, movement of the respective cartridge 3 out of the cartridge storage unit 5, which is preceded by an upward, such as vertical, movement of the cartridge 3 within the cartridge storage unit 5. In order for the cartridge 3 to reach the above noted, first plane 13, another upward movement of the cartridge 3, this time together with the cartridge drive unit 8, can be provided, as shown in FIG. 5d. During this upward movement, in some embodiments, the operative coupling between the cartridge 3 and the cartridge drive unit 8 is established. As a result, the cartridge 3 is in the drive location 7 for a possible connection to a receptacle 10. All in all, FIG. 5 shows that although the transfer locations 6 are each aligned in an above noted first plane 13, it is well possible to use vertical movements for the optimal use of the existing workspace.

According to one embodiment it is proposed, that the unit operation station 2 comprises a temperature control device to control the temperature of the cartridges 3, which are being stored in the cartridge storage unit 5. By using a temperature control device to control the temperature of the cartridges 3 that are stored in the cartridge storage unit 5, liquids preconfigured for the respective unit operation may be stored within the cartridge storage unit 5. As explained above, this provides flexibility in terms of automatization, as the liquids may be stored within the cartridge 3 until they are needed. Additionally, the cartridge storage unit 5 may comprise means to record the temperature within the cartridge storage unit 5 to verify that a pre-defined temperature is maintained.

Additionally or alternatively, the cartridge drive unit 8 may comprise a temperature control device to control the temperature of the cartridge fluidic structure 4.

Another teaching which is of equal importance relates to an integrated bioprocessing system 1 for performing a bioprocess on liquid immune cell cultures, wherein the bioprocess comprises a sequence of processing steps, wherein the sequence of processing steps comprises at least one unit operation, and that the integrated bioprocessing system 1 comprises a number of unit operation stations 2 for performing the unit operations.

Essential for this third teaching is, that each of the unit operation stations 2 are being designed according the second teaching. Moreover, in some embodiments, the integrated bioprocessing system 1 is designed to perform the method according to the first teaching. As a result, reference may be made to the first two teachings in order to avoid redundancies.

According to various embodiments, at least part of the unit operation stations 2 and/or at least part of the cartridges 3 and/or part of the receptacles 10 are widely identical to each other. It has been explained before, which benefits go along with this kind of standardization.

According to one embodiment it is proposed, that the cartridges 3, that are being stored in a cartridge storage unit 5, are preconfigured such that the unit operation station 2 is able to perform only one unit operation dedicated to this unit operation station 2. In addition or as an alternative it is proposed, that the cartridges 3, that are being stored in the cartridge storage unit 5, are preconfigured such that the unit operation station 2 is able to perform different unit operations depending on the configurations of the cartridges 3. The combination of FIG. 1 and FIG. 2 shows, that those two concepts may easily be combined in a single integrated bioprocessing system 1.

According to another embodiment it is proposed, that the integrated bioprocessing system 1 comprises a number of receptacles 10, that each have been preconfigured with a receptacle fluidic structure 11 for containing a liquid, such as an immune cell culture, and a global transport mechanism 12 for the transport of the receptacles 10.

The receptacle 10 comprises a flat receptacle carrier 38 to receive a receptacle fluidic structure 11. As will be explained later, the global transport mechanism 12 can be a conveyor mechanism 39 and/or a robotic mechanism 40. After a liquid has been transferred from a receptacle fluidic structure 11 of a receptacle 10 and/or after waste has been transferred to the receptacle fluidic structure 11 of a receptacle 10, the respective receptacle 10, including the receptacle fluidic structure 11 and any liquid contained therein, may be transferred to a central waste storage 24. The receptacles 10 disposed in the central waste storage 24 may be periodically withdrawn by an operator.

Receptacles 10 preconfigured with a receptacle fluidic structure 11 for containing a liquid that are transported by the global transport mechanism 12 may be supplied from a central supply storage 23. As depicted, the integrated bioprocessing system 1 may comprise more than one central supply storage 23 and/or central waste storage 24. The global transport mechanism 12 can be designed to transport receptacles 10 to and from the respective central supply storage 23 and/or central waste storage 24.

To retrieve a receptacle 10, in some embodiments comprising a receptacle fluidic structure 11 containing a liquid from the enclosure 34 and/or to introduce a receptacle 10, in some embodiments comprising a receptacle fluidic structure 11 containing a liquid into the enclosure 34 of the integrated bioprocessing system 1, the integrated bioprocessing system 1 comprises at least one, in some embodiments more than one, input-output location 41.

According to one embodiment it is proposed, that the global transport mechanism 12 comprises a conveyor mechanism 39, such as a belt conveyor mechanism, a roller conveyor mechanism, a ball conveyor system, and/or, that the global transport mechanism 12 comprises a robotic mechanism 40. It can be that the global transport mechanism 12 comprises at least one robotic mechanism 40. However, it is also possible that two or more robotic mechanisms 40 are provided to increase the robustness by adding redundancy in case one robotic mechanism 40 fails. An above noted conveyor mechanism 39 is particularly simple as receptacles 10 only need to provide a flat tray structure to be transported in a standardized way. Accordingly, transport of the receptacles 10 by a conveyor mechanism 39 can be provided. However, additionally or alternatively, transport of the receptacles, may at least in part also be realized by the robotic mechanism 40. The possibility to use two different transport mechanisms 39, 40 contributes to the redundancy of the system and increases the process robustness.

According to one embodiment it is proposed, that the receptacle fluidic structure 11 comprises a receptacle liquid container 46 and a receptacle tube set 49 connected to the receptacle liquid container 46. Here it can be that at least part of the receptacle fluidic structure 11 is composed of a single-use material. It can be that all elements of the receptacle fluidic structure 11 that are configured to be in contact with a liquid are composed of a single-use material. As already mentioned, in some embodiments, that the receptacle 10 comprises a tray with a basically flat receptacle carrier 38 to receive the components of the receptacle fluidic structure 11. The combination of FIG. 3 and FIG. 4 shows, that this tray like, flat structure is especially advantageous, if an above noted conveyor mechanism 39 is applied. In various embodiments, and as for example depicted in FIG. 3, the receptacle liquid container 46 is oriented on the receptacle carrier 38 in a basically horizontal position. Alternatively, the receptacle liquid container 46 may also be held vertically within the receptacle 10, for example by attaching it to a pole or the like so that it may be transported in a hanging manner. Further, using a tray to receive the components of the receptacle fluidic structure 11 ensures that any liquid that may leak from the receptacle fluidic structure 11, for example in case of a breach in the receptacle liquid container 46 and/or in the receptacle tube set 49, is contained within the receptacle 10 and does not contaminate other elements of the integrated bioprocessing system 1.

As noted above, the integrated bioprocessing system 1 is a closed bioprocessing system designed to perform a closed bioprocess including a number of closed unit operations, such that all liquids involved in the bioprocess are being kept within a closed inner volume. For this it can be that at least part of the liquid handling involved in the respective unit operation is being performed within tubes. Reference may be made to the explanations given above with respect to the closed bioprocess.

As described above, for realizing the closed bioprocess in an automated way, it is proposed, that the integrated bioprocessing system 1 comprises a tube connection system 17, such as a tube welding system 17a, which is designed to perform a closed connection process. In this connection process, a cartridge transfer tube 15 of a cartridge 3, located at a cartridge drive location 7, and a receptacle transfer tube 16 of a receptacle 10, located at a transfer location 6, are being connected automatically. The tube connection system 17 can comprise a tube connection head 17b, such as a tube welding head 17c, and a connection handling mechanism for positioning the tube connection head 17b. The function of the connection handling mechanism may well be provided by the global transport mechanism 12, such as by the robotic mechanism 40.

As shown in FIG. 8, the tube connection system 17 is designed to, within the connection process, perform a step of arranging the tubes 15, 16 to be connected relative to the rest of the connection system 17 (FIG. 8a)) and a subsequent step of welding the tubes 15, 16 to be connected, in particular by the tube welding head 17c (FIG. 8d)). In various embodiments, the tube connection system 17 is designed to also perform a step of trimming the tubes 15, 16 to be connected, before performing the step of welding, which is shown by the sequence of FIG. 8b) to FIG. 8c).

In various embodiments, the tube welding head 17c is positioned relative to the tubes 15, 16 to be connected, which tubes are protruding out of the respective anchor points 18, 20 towards each other, as described above. The result is the arrangement of the tubes 15, 16 relatively to the rest of the connection system 17, as is schematically depicted by the sequence of FIG. 8a) and FIG. 8b). Here it can be that the tubes 15, 16 are also being arranged relatively to each other. As depicted in FIG. 8a), the tubes 15, 16 may be arranged to overlap at least partially. After arranging the tubes 15, 16, a trimming step can be performed by the welding system 17a. This trimming step is schematically depicted by the sequence of FIG. 8b) to FIG. 8c). The trimming step can include cutting each of the tubes 15, 16 at one location. In various embodiments, the trimming step is performed by a heated blade 50 that is moved in a cutting direction, such as vertically, here in the z-direction, to cut each of the transfer tubes 15, 16 at one position. In various embodiments, the blade 50 is part of the tube welding head 17c and/or operated by the tube welding head 17c. Accordingly, it can be that the tube welding head 17c also comprises a device to heat the blade 50. Alternatively, other measures to cut the tubes 15, 16 are imaginable as well, for example a laser cutting device or the like. In various embodiments, the tubes 15, 16 are cut simultaneously. However, the tubes 15, 16 may also be cut sequentially.

After the tubes 15, 16 are cut, the parts of the respective tubes 15, 16 to be connected are moved relatively to each other (FIG. 8c)) in such a way that the tube ends to be welded are aligned. Relative movement of the tubes 15, 16 may be realized by moving the receptacle anchor point 20 and/or the cartridge anchor point 18. Then, in some embodiments, the anchor points 18, 20 are to be considered part of the tube connection system 17.

In various embodiments, the above noted, relative movement between the tubes 15, 16 is a linear movement in a horizontal direction, here in the y-direction. This is indicated by the arrow in FIG. 8c). It can be that by the relative movement, the respective anchor points 18, 20 are aligned, as indicated in FIG. 8d). It can be that the blade 50 remains in position between the cut tubes 15, 16 until the tubes 15, 16 have been aligned. After alignment, the blade 50 is moved upwards, such as vertically, here in the z-direction, and the tube ends are merged together by welding based on the remaining heat of the blade 50. After welding, an integrity test may be performed to verify that the weld has been correctly established.

In various embodiments, the tube welding system 17a is configured to perform the integrity test on the weld location 51 after connecting the respective transfer tubes 15, 16 to verify that the inner volume remains closed after the closed connection process was performed. For the definition of “closed inner volume”, reference is made to the definition given above. In case the integrity test fails and it is not verified that the inner volume is closed, the tube welding system 17a may perform a disconnecting process, as mentioned above, and subsequently perform another closed connection process as also described above.

The integrated bioprocessing system 1 can include an electronic process control 22 for coordinating the at least one bioprocess and that the electronic process control 22 is designed to have at least two bioprocesses to be performed at least partly simultaneously, and/or to have at least two unit operation stations 2 perform unit operations on different liquid immune cell cultures at least partly simultaneously.

As noted above, the sequence of processing steps comprises at least one service operation, which is being performed within at least one service station of the integrated bioprocessing system 1, separately from the unit operation stations 2. As also noted above, the at least one service operation may comprise an incubation step, a sampling step, a media fill step or the like. The at least one service station can include at least one service transfer location, to which the receptacles 10 may be transported by the global transport mechanism 12 for performing the service operation.

In a particularly compact design, at least part of the service transfer locations are arranged in a second plane 42, which second plane 42 is offset the first plane 13. The receptacles 10 may then be transported by the global transport mechanism 12 to and from the service transfer locations in this second plane 42 as well. The global transport mechanism 12 can also serve to transport the receptacles 10 between the first plane 13 and the second plane 42. Alternatively, for vertical transport between the first plane 13 and the second plane 42, an elevator system 43 or the like may be used. This may be taken from a combination of FIG. 1 and FIG. 4.

According to one embodiment it is proposed, that the integrated bioprocessing system 1 comprises an enclosure 34, which houses at least part of the unit operation stations 2. As shown in FIG. 1, the enclosure 34 may be realized as a simple housing, that houses at least part of the components of the integrated bioprocessing system 1, in particular the unit operation stations 2 and/or the service stations. It can be (not shown), that the integrated bioprocessing system 1 comprises an environmental condition control, which guarantees predefined environmental conditions within the enclosure 34.

To clarify various embodiments, the first processing steps of a bioprocess for manufacturing genetically modified CAR-T cells from a liquid immune cell culture will be described in the following.

As may best be seen from FIG. 4, a receptacle 10 containing an initial liquid immune cell culture is introduced into the integrated bioprocessing system 1 by an operator at an input-output location 41. From the input-output location 41, the receptacle 10 is transferred to a transfer location 6 of a unit operation station 2 by the global transport mechanism 12. In the example given, the unit operation station 2 is being preconfigured to the enrichment processing step by performing the standard routine on the respective cartridge 3, that has been preconfigured accordingly. In detail, the preconfigured cartridge 3 is being transported from the cartridge storage unit 5 to the cartridge drive unit 8.

In various embodiments, the unit operation of the enrichment processing step at least comprises a counter-flow centrifugation unit operation step. However, as mentioned above, instead of counter-flow centrifugation, also acoustic separation may be used. For counter-flow centrifugation, the cartridge fluidic structure 4 of the respective cartridge 3 comprises a counter-flow centrifuge element to receive a liquid to be centrifuged. Accordingly and as mentioned above, the counter-flow centrifuge element can be made of a single-use material. Before, after or during the transport of the receptacle 10 to the transfer location 6 of the unit operation station 2, the cartridge 3 of the unit operation station 2, that comprises a cartridge fluidic structure 4 that has been preconfigured to the enrichment processing step, is brought into operative coupling with the cartridge drive unit 8 within the above noted standard routine. In detail, the counter-flow centrifuge element of the cartridge fluidic structure 4 is brought into operative coupling with a mechanical interface of the cartridge drive structure 31 as part of the operative coupling. This mechanical interface may be a rotor that is used to rotate the counter-flow centrifuge element. As mentioned above, the rotor can be designed to be used multiple times.

After the receptacle 10 containing the liquid immune cell culture has been transported to the transfer location 6 of the unit operation station 2 and the cartridge 3 has been transported to the drive location 7 within the standard routine, the receptacle transfer tube 16 and one of the cartridge transfer tubes 15 are connected by welding in the closed connection process (FIG. 6a).

Next, the liquid immune cell culture is transferred via the welded connection from the receptacle 10 to the counter-flow centrifuge element of the cartridge 3 using a cartridge tube set 44 and a peristaltic pump 30 to transfer the liquid immune cell culture within the tube set (FIG. 3). Again, the respective interface for engaging the peristaltic pump 30 is provided by the drive structure 31.

Next, counter-flow centrifugation is carried out on the liquid immune cell culture by the counter-flow centrifuge of the cartridge 3. For this, the counter-flow centrifuge element is fluidically connected to a cartridge liquid container 45 that may receive the waste liquid from the counter-flow centrifugation unit operation step. Alternatively, the counter-flow centrifuge element is fluidically connected to a receptacle 10 provided in a transfer location 6 with the receptacle 10 comprising a receptacle fluidic structure 11 not yet containing a liquid. The receptacle 10 may be transported to the transfer location 6 by the global transport mechanism 12 and may be provided from a central supply storage 23.

Here it should be noted, that at least part of the liquids required at a respective unit operation station 2 for performing a unit operation may also be supplied from the media fill service station 27 after the respective liquid has been transferred into a receptacle 10 at a media fill location 28 of the media fill service station 27 as described above. In this case, the receptacle 10 is transported to a transfer location 6 of the particular unit operation station 2 by the global transport mechanism 12 and the connection between the cartridge 3 and the receptacle 10 is established in a connection process as described above.

Optionally, a washing unit operation step may be carried out after the counter-flow centrifugation unit operation step (not depicted). For this, the cartridge 3 may comprise at least one cartridge liquid container 45 with a washing liquid. The washing liquid is transferred from the cartridge liquid container 45 to the counter-flow centrifuge element. Alternatively, the washing liquid may be provided from the media fill service station 27. In the latter case, the washing liquid is transferred from a media storage container to a receptacle liquid container 46 as described above. Afterwards, the receptacle 10 is transported by the global transport mechanism 12 to a transfer location 6 of the unit operation station 2 and connected to the cartridge 3 in the closed connection process also described above.

Again, the waste liquid from the washing unit operation step is transferred to a cartridge liquid container 45. Alternatively, the waste liquid is transferred to a receptacle 10 provided in a transfer location 6. In various embodiments, the same liquid container or receptacle 10 that was also used to receive the waste liquid from the centrifugation of the liquid immune cell culture is used to receive the waste liquid from the washing unit operation step. However, in an alternative embodiment, the cartridge fluidic structure 4 may comprise different cartridge liquid containers 45 to receive the waste liquid. After transfer of the liquid waste of the washing unit operation step, the now purified liquid immune cell culture may be eluted from the counter-flow centrifuge element and transferred to a second receptacle 10 that is provided in a second transfer location 6 of the unit operation station 2 (FIG. 3). Again, a connection between the receptacle transfer tube 16 of the second receptacle 10 and a cartridge transfer tube 15 of the cartridge 3 is established in a closed connection process. For elution, the cartridge 3 may comprise another liquid container that comprises an elution liquid like culture medium or the like (not depicted). The purified T cells are eluted from the counter-flow centrifuge to a receptacle liquid container 46 of the second receptacle 10.

After all unit operation steps of the unit operation of the enrichment processing step have been performed, the welded connections between the receptacle transfer tubes 16 and the respective cartridge transfer tubes 15 of the cartridge 3 are disconnected in the disconnecting process. Afterwards, the cartridge 3 is transferred from the cartridge drive unit 8 to the cartridge waste storage unit 21 as described above.

After disconnection, the first receptacle 10, now comprising an empty receptacle liquid container 46, may be transported by the global transport mechanism 12 to the central waste storage 24 to be discarded. The second receptacle 10, now containing the partly processed liquid immune cell culture, is transferred by the global transport mechanism 12 to the unit operation station 2, which is preconfigured for the selection processing step.

During the selection processing step, a certain subtype of the liquid immune cells within the liquid immune cell culture is enriched. In various embodiments, a subtype of T cells is enriched. In various embodiments, a subtype of T cells expressing the antigen CD4, CD8 or CD62L is enriched. In a proposed embodiment, the unit operation of the selection processing step comprises at least a magnetic separation unit operation step.

For the selection processing step, the receptacle 10 containing the liquid immune cell culture after the enrichment processing step, is now transported to a transfer location 6 of a particular unit operation station 2, which is preconfigured for the selection processing step by preconfiguration of one of its cartridges 3. As explained for the enrichment processing step, the cartridge 3 for the unit operation of the selection processing step, which is stored in the cartridge storage unit 5 of one of the unit operation stations 2, has been moved into the drive location 7 of this particular unit operation station 2.

After the connection process for establishing a fluidic connection between the receptacle 10 and the cartridge 3, from a cartridge liquid container 45, a liquid comprising magnetic beads that have been coated with an antibody directed against the antigen on the T cell surface, is transferred into the receptacle 10 containing the liquid immune cell culture in a liquid addition unit operation step. Again, the liquid connection is established by the welding system 17a connecting the respective transfer tubes in a closed manner.

In a next step, the receptacle 10 comprising the liquid immune cell culture and the magnetic beads is transported to an incubator service station 25 that is located in the second plane 42. The incubator service station 25 comprises at least one incubator service transfer location 47 to receive the receptacle 10. In an embodiment and as depicted in FIG. 4, the incubator service station 25 may be designed as a drawer system. Alternatively, the incubator service station 25 may also be designed as a shelf system. However, also other configurations are imaginable. Again, the global transport mechanism 12 is used for the transport and an elevator system 43 may be used to transfer the receptacle 10 from the first plane 13 to the second plane 42 (FIG. 4). It should be noted, that while the incubator service station 25, including the at least one incubator service transfer location 47, is located in the second plane 42 in the example described here, it is also well possible that at least one incubator service station 25 may be located in the first plane 13.

At the incubator service station 25, the receptacle 10 is subjected to defined process conditions for a predefined amount of time in the incubation step. The incubation step is carried out to enable binding of the target immune cells to the magnetic particles via the antigen-antibody bond. Hence, T cells comprising a certain antigen corresponding to the antibody coated to the magnetic particles, are bound to the magnetic particles.

After the incubation step, the receptacle 10 is being transported from the incubator service station 25 back to the unit operation station 2, that is preconfigured for the selection processing step. Here, the magnetic separation unit operation step and several subsequent steps are being carried out according to the same inventive method.

While the example given above describes a sequence of processing steps to be carried out to manufacture genetically modified CAR T-cells it should be noted that this example only serves to clarify some embodiments. The exact sequence of processing steps, unit operation steps, unit operations involved and/or layout of the cartridge fluidic structures 4 may well and easily be adapted to the specific bioprocess requirements. For example, and as mentioned partly above, additional liquid containers and/or tube sets may be located within a cartridge 3 where required and/or other technology might be used to perform any of the unit operations mentioned. Also, the sequence of processing steps might vary with some processing steps being repeated and/or omitted. Also, any type, number and sequence of unit operation steps might be repeated and/or omitted. As mentioned above, the number, and arrangement of unit operation stations 2, and/or the type and number of robotic mechanisms and/or the number and arrangement of service stations within the integrated bioprocessing system 1 may also be flexibly adapted.

The flexibility in terms of type, number and/or arrangement of the mentioned elements also contributes to the robustness of the integrated bioprocessing system 1, which is important for process reliability, particularly to ensure that each liquid immune cell culture may still be processed, even if an element of the integrated bioprocessing system 1 fails. For example, and as mentioned above, including a number of unit operation stations 2 that are configured to carry out the same unit operation, provides redundancy in case that one unit operation station 2 fails. This way, the electronic process control 22, described in detail above, could easily assign the respective liquid immune cell culture to a different unit operation station 2. Here it is to be noted further, that a sampling step may be carried out between any unit operation step and/or incubation step to determine a certain feature of the liquid immune cell culture. For example, the receptacle 10 containing the liquid immune cell culture may be transported from the incubator service station 25 to a sampling service station 26 to derive a sample to analyze the current cell viability. After sampling, the receptacle 10 containing the liquid immune cell culture may be transported back to the incubator service station 25 to continue incubation and/or expansion or it may be transferred to a unit operation station 2 to continue the processing step currently carried out or start the next processing step at a different unit operation station 2.

The sequence of processing steps and/or the sequence of unit operation steps may be adapted based on the analysis results of the sampling steps. For example, a washing step might be included and/or repeated in the enrichment processing step, if the number of impurities is determined to be above a predefined threshold. Further, the duration of an incubation step may be adjusted based on the measurement of the viable cell concentration of the liquid immune cell culture.

1-46. (canceled)

47. A method for performing a bioprocess on liquid immune cell cultures comprising a sequence of processing steps, wherein the at least one bioprocess is being performed on an integrated bioprocessing system, wherein the sequence of processing steps comprises at least one unit operation, which is being performed within a unit operation station of the integrated bioprocessing system,
wherein cartridges, that have been preconfigured with a cartridge fluidic structure with respect to at least one unit operation, are being stored in a cartridge storage unit of the unit operation station and that for performing a unit operation, in a standard routine, one of the cartridges is being transported from a storage transfer location in the cartridge storage unit of the unit operation station to a drive location of a cartridge drive unit of the unit operation station by a local transport mechanism of the unit operation station and is being brought into operative coupling with the cartridge drive unit.

48. The method according to claim 47, wherein the at least one unit operation station comprises at least one transfer location, to which receptacles, that have been preconfigured with a receptacle fluidic structure for containing a liquid are being transported by a global transport mechanism of the integrated bioprocessing system, in order to transfer liquid between a cartridge, which has been transported to the drive location, and the receptacle.

49. The method according to claim 47, wherein the integrated bioprocessing system comprises a number of unit operation stations.

50. The method according to claim 47, wherein the at least one bioprocess is a closed bioprocess, such that all liquids involved in the respective unit operation are being kept within a closed inner volume.

51. The method according to claim 47, wherein for the transfer of liquid between the cartridge and the receptacle, a cartridge transfer tube of the cartridge and a receptacle transfer tube of the receptacle are being connected in a closed connection process by a tube connection system.

52. The method according to claim 47, wherein at least two said bioprocesses are being performed at least partly simultaneously by the integrated bioprocessing system.

53. The method according to claim 47, wherein the sequence of processing steps comprises at least one service operation, which is being performed within at least one service station of the integrated bioprocessing system, separately from the unit operation stations.

54. A unit operation station of an integrated bioprocessing system for performing at least one bioprocess on liquid immune cell cultures, wherein the sequence of processing steps comprises at least one unit operation, which is being performed within a unit operation station, wherein the unit operation station comprises a number of units, wherein one of the units is a cartridge storage unit for receiving cartridges, which cartridges may each be preconfigured with a cartridge fluidic structure with respect to at least one unit operation, that another one of the units is a cartridge drive unit with a drive structure, that the unit operation station comprises a local transport mechanism and that in a standard routine, the local transport mechanism may transport one of the preconfigured cartridges from a storage transfer location in the cartridge storage unit to a drive location of the cartridge drive unit, wherein the cartridge may be brought into an operative coupling with the cartridge drive unit to perform the respective unit operation.

55. The unit operation station according to claim 54, wherein the cartridge fluidic structure comprises a cartridge liquid container such as a liquid bag, a functional device such as a separation device and a cartridge tube set for fluidically connecting those components to each other and/or to other components.

56. The unit operation station according to claim 54, wherein the drive structure provides one or more of a mechanical interface, a fluidic interface and an electrical interface to the cartridge, which cartridge is located in the drive location.

57. The unit operation station according to claim 54, wherein the transport of one of the cartridges from the cartridge storage unit to the cartridge drive unit involves one or more of a sideways movement of the respective cartridge out of the cartridge storage unit and an upward movement of the cartridge to position the cartridge in the drive location for a possible connection to a receptacle.

58. The unit operation station according to claim 54, wherein the unit operation station comprises a temperature control device to control the temperature of the cartridges, which are being stored in the cartridge storage unit.

59. An integrated bioprocessing system for performing a bioprocess on liquid immune cell cultures, wherein the bioprocess comprises a sequence of processing steps, wherein the sequence of processing steps comprises at least one unit operation, and that the integrated bioprocessing system comprises a number of unit operation stations for performing the unit operations, wherein each of the unit operation stations are being designed according to claim 54.

60. The integrated bioprocessing system according to claim 59, wherein at least two of the unit operation stations are identical to each other with the exception of the respective cartridge drive units, which may be preconfigured to the respective cartridges.

61. The integrated bioprocessing system according to claim 59, wherein the cartridges, that are being stored in a cartridge storage unit, are preconfigured such that the unit operation station is able to perform only one unit operation dedicated to this unit operation station, or, that the cartridges, that are being stored in a cartridge storage unit, are preconfigured such that the unit operation station is able to perform different unit operations depending on the configurations of the cartridges.

62. The integrated bioprocessing system according to claim 59, wherein the integrated bioprocessing system comprises a number of receptacles, that each have been preconfigured with a receptacle fluidic structure for containing a liquid and a global transport mechanism for the transport of the receptacles.

63. The integrated bioprocessing system according to claim 59, wherein the global transport mechanism comprises one of a conveyor mechanism, a belt conveyor mechanism, a roller conveyor mechanism, a ball conveyor system, and a robotic mechanism.

64. The integrated bioprocessing system according to claim 59, wherein the integrated bioprocessing system comprises a tube connection system which is designed to perform a closed connection process, connecting a cartridge transfer tube of a cartridge, located at a cartridge drive location, and a receptacle transfer tube of a receptacle, located at a transfer location.

65. The integrated bioprocessing system according to claim 59, wherein the integrated bioprocessing system comprises an electronic process control for coordinating the at least one bioprocess and that the electronic process control is designed to have at least two bioprocesses to be performed at least partly simultaneously, or to have at least two unit operation stations perform unit operations on different liquid immune cell cultures at least partly simultaneously.

66. The integrated bioprocessing system according to claim 59, wherein the integrated bioprocessing system comprises an enclosure, which houses at least part of the unit operation stations.
